(19) United States
(12) Patent Application Publication
(10) Pub. No.: US 2016/0252504 A1
(43) Pub. Date: Sep. 1, 2016

(54) TARGET TESTING DEVICE, TARGET TESTING KIT, TARGET TESTING METHOD, TRANSFER MEDIUM, AND METHOD FOR PRODUCING TARGET TESTING DEVICE

(71) Applicant: Rie KOBAYASHI, Kanagawa (JP)

(72) Inventor: Rie KOBAYASHI, Kanagawa (JP)

(21) Appl. No.: 15/054,654

(22) Filed: Feb. 26, 2016

(30) Foreign Application Priority Data
Feb. 27, 2015 (JP) 2015-037576
Feb. 23, 2016 (JP) 2016-032354

(51) Int. Cl. G01N 33/543; G01N 33/558; B32B 37/12; G01N 33/545

(52) U.S. Cl. CPC G01N 33/54366; G01N 33/545; G01N 33/558; B32B 37/1207; B32B 2037/1215; B32B 2307/728; B32B 2398/20; B32B 2535/00

(57) ABSTRACT

A target testing device including a porous flow path member as a flow path for a testing target liquid to flow through, a first-antibody supplying portion configured to supply a first antibody to the testing target liquid flowing through the flow path member, and a second-antibody immobilized portion that is a resin shaped body disposed at a position on the flow path downstream from the first-antibody supplying portion in a manner to contact the flow path member. The first antibody contains a label and is bindable with any target substance possibly contained in the testing target liquid. A second antibody bindable with at least one of the first antibody and the target substance is immobilized to a surface of the shaped body contacting the flow path member by covalent binding with the shaped body.

### CROSS-REFERENCES TO RELATED APPLICATIONS

The present application claims priority under 35 U.S.C. §119 to Japanese Patent Application No. 2015-037576, filed Feb. 27, 2015, and Japanese Patent Application No. 2016-032354, filed Feb. 23, 2016. The contents of which are incorporated herein by reference in their entirety.

### BACKGROUND OF THE INVENTION

1. Field of the Invention

The present disclosure relates to target testing devices, target testing kits, target testing methods, transfer media, and methods for producing target testing devices.

2. Description of the Related Art

Hitherto, target testing devices in which a flow path for flowing an analyte is formed have been used for testing analytes such as bloods, DNAs, foods, and beverages. Examples of the target testing devices include a target testing device including a sample pad as a liquid receiving portion for receiving a testing target liquid, a conjugate pad in which the testing target liquid supplied from the sample pad undergoes a reaction, and a membrane film for flowing the testing target liquid supplied from the conjugate pad (see, e.g., Japanese Unexamined Patent Application

Publication No. 2010-256309). The conjugate pad contains a labeled antibody obtained by labeling an antibody with a pigment, and when supplied with the testing target liquid from the sample pad, makes an antigen (i.e., a target substance) contained in the testing target liquid react with the labeled antibody and supplies the reacted antigen to the membrane film. Meanwhile, a detecting portion of the membrane filter is previously coated with an antibody (i.e., a capture antibody) for capturing the antigen. The antigen contained in the testing target liquid supplied from the conjugate pad is captured at the detecting portion in the state of being bound with the labeled antibody. As a result, the detecting portion develops a color. This makes it possible to visually observe or measure the degree of color development and qualitatively or quantitatively measure the antigen in the testing target liquid.

Incidentally, when using a target testing kit for a quick in-vitro diagnosis, there is a need for saving the time taken for the testing in order to reduce burdens on the doctor and the patient. Hence, Japanese Unexamined Patent Application Publication No. 2010-256309 mentioned above discloses adjustment of a water absorbing speed of a synthetic fiber constituting the conjugate pad to increase a testing-target-liquid spreading speed and save the testing time.

### SUMMARY OF THE INVENTION

The present invention has an object to provide a target testing device that makes it possible to realize a highly sensitive measurement and obtain a clear judgment line even when a flow path member of the target testing device is changed.

A target testing device of the present invention as means for solving the problem described above includes a porous flow path member as a flow path for a testing target liquid to flow through, a first-antibody supplying portion configured to supply a labeled antibody to the testing target liquid flowing through the flow path member, and a second-antibody immobilized portion that is a resin shaped body disposed at such a position on the flow path as downstream from the first-antibody supplying portion in a manner to contact the flow path member. The labeled antibody contains a label and a first antibody bindable with any target substance that is possibly contained in the testing target liquid. A second antibody bindable with at least one of the first antibody and the target substance is immobilized to such a surface of the shaped body as contacting the flow path member by covalent binding with the shaped body.

According to the present invention, it is possible to solve the various conventional problems and provide a target testing device that makes it possible to realize a highly sensitive measurement and obtain a clear judgment line even when a flow path member of the target testing device is changed.

### BRIEF DESCRIPTION OF THE DRAWINGS

FIG. 1 is a top view of a target testing device according to an embodiment of the present invention;

FIG. 2 is a cross-sectional view of a target testing device according to an embodiment of the present invention, the view being parallel with the longer direction of the target testing device;

FIG. 3 is a partial enlarged view depicting a portion at which a first second-antibody immobilized portion and a flow path member contact each other;

FIG. 4 is a partial enlarged view depicting a portion at which a second second-antibody immobilized portion and a flow path member contact each other;

FIG. 5 is a conceptual diagram of a membrane of a conventional target testing device;

FIG. 6 is a cross-sectional view illustrating a layered state of a transfer medium used for a target testing device;

FIG. 7 is a conceptual diagram of a testing kit according to an embodiment of the present invention;

FIG. 8A is a top view of a target testing device according to a comparative example; and

FIG. 8B is a cross-sectional view of FIG. 8A, the view being parallel with the longer direction of FIG. 8A.

### DETAILED DESCRIPTION OF THE INVENTION

### (Target Testing Device)

A target testing device of the present invention includes a porous flow path member as a flow path for a testing target liquid to flow through, a first-antibody supplying portion configured to supply a labeled antibody to the testing target liquid flowing through the flow path member, and a second-antibody immobilized portion that is a resin shaped body disposed at such a position on the flow path as downstream from the first-antibody supplying portion in a manner to contact the flow path member. The labeled antibody contains a label and a first antibody bindable with any target substance that is possibly contained in the testing target liquid. A second antibody bindable with at least one of the first antibody and the target substance is immobilized to such a surface of the shaped body as contacting the flow path member by covalent binding with the shaped body.

The target testing device of the present invention is based on a finding from a conventional target testing device in which reagents such as a labeled antibody and a capture antibody and reagents such as a labeling indicator and a detecting indicator are immobilized to fibers in the flow path member, and hence the flow path member that is made of a material optionally selected for improving an analyte spreading speed may have an excessively strong interaction with the reagents such as the labeled antibody and the labeling indicator and may not be able to spread the reagents, or may have an excessively weak interaction with the reagents such as the antibody and the detecting indicator and may not be able to immobilize the analyte that is captured.

The target testing device of the present invention is also based on a finding from a conventional target testing device in which a capture antibody is present diffusively in a hydrophilic porous material of which the flow path member is made because a test line and a control line are formed by directly applying a liquid in which the capture antibody is dissolved over the flow path member, but a color developed by labeling particles (e.g., gold particles) of the labeled antibody bound with the capture antibody present in the hydrophilic porous material cannot actually be sensed due to light scattering, i.e., most of the capture antibody is not used effectively.

### <Flow Path Member>

The flow path member of the target testing device is not particularly limited so long as the flow path member is a porous member capable of flowing the testing target liquid through the porous member. Examples of the flow path member include a hydrophilic porous material. The flow path member made of the hydrophilic porous material contains voids, and the flow path is formed when the testing target liquid flows through the voids. It is preferable that cells be present in the hydrophilic porous material and that the cells be linked together to form a continuous cell. The continuous cell is distinguished from independent cells that are not linked together. The continuous cell has a function of sucking in a liquid by a capillary action or letting a gas pass through the continuous cell because the continuous cell has small holes in the walls between the cells. The flow path member needs no external actuating device such as a pump because the flow path member is configured to deliver the testing target liquid by utilizing a capillary action through the voids.

A spreading speed in the flow path member has no particular limit, and may be appropriately selected according to the purpose.

The hydrophilic porous material is not particularly limited, and an arbitrary hydrophilic porous material may be selected according to the purpose. However, a material having hydrophilicity and a high voidage is preferable. The hydrophilic porous material refers to a porous material that is easily permeable by an aqueous solution. The hydrophilic porous material is referred to as being easily permeable when in a water permeability evaluation test in which 0.01 mL of pure water is chopped onto a surface of a plate-shaped test piece of the hydrophilic porous material dried at 120° C. for 1 hour, 0.01 mL of the pure water completely permeates the test piece in 10 minutes.

The voidage of the hydrophilic porous material has no particular limit, and may be appropriately selected according to the purpose. However, the voidage is preferably 40% or higher but 90% or lower, and more preferably 65% or higher but 80% or lower. When the voidage is 90% or lower, the flow path member can maintain the strength as the flow path member. When the voidage is 40% or higher, permeability of the testing target liquid is not influenced. The voidage can be calculated according to a calculation formula 1 below based on a basis weight (g/m²) and an average thickness (μm) of the hydrophilic porous material and the specific gravity of the component of the hydrophilic porous material.

Voidage %={1−[basis weight (g/m²)/average thickness (μm)/specific gravity of the component]}×100 [Calculation formula 1]

The hydrophilic porous material is not particularly limited, and an arbitrary hydrophilic porous material may be selected according to the purpose. Examples of the hydrophilic porous material include filter paper, regular paper, high-quality paper, watercolor paper, Kent paper, synthetic paper, synthetic resin film, special-purpose paper with a coat layer, fabric, fiber product, film, inorganic substrate, and glass.

Examples of the fabric include artificial fiber such as rayon, bemberg, acetate, nylon, polyester, and vinylon, natural fiber such as cotton and silk, blended fabric of these, or non-woven fabric of these.

Among these, filter paper is preferable because filter paper has a high voidage and a favorable hydrophilicity. When the target testing device is used as a biosensor, the filter paper is preferable as a static bed of paper chromatography.

The shape of the hydrophilic porous material is not particularly limited, and may be appropriately selected according to the purpose. However, a sheet shape is preferable.

The average thickness of the hydrophilic porous material has no particular limit, and may be appropriately selected according to the purpose. However, the average thickness is preferably 0.01 mm or greater but 0.3 mm or less. When the average thickness is 0.01 mm or greater, the hydrophilic porous material can maintain the strength as the flow path member. When the average thickness is 0.3 mm or less, the amount of the testing target liquid needed can be saved. In the present embodiment, the thickness can be defined as the length of an article in a direction perpendicular to an interface at which a base material and the flow path member contact each other.

### <First-Antibody Supplying Portion>

The first-antibody supplying portion is not particularly limited, and an arbitrary first-antibody supplying portion may be selected according to the purpose so long as the first-antibody supplying portion can supply a first antibody to the flow path member. Examples of the first-antibody supplying portion include a structure for the first antibody to be dropped into the flow path using separately provided device and tool, and a structure configured to supply the first antibody from a first-antibody spreading member containing the first antibody and laminated over the flow path. Of these, the structure including the first-antibody spreading member is preferable.

### <<First-Antibody Spreading Member>>

The first-antibody spreading member is not particularly limited, and a known material may be selected according to the purpose so long as the material can support the first antibody in a state capable of spreading the first antibody. Examples of the material include cellulose filter paper, glass fiber, and non-woven fabric.

A method for supporting the first antibody over the first-antibody spreading member is not particularly limited, and an arbitrary method may be selected according to the purpose. Examples of the method include a method for impregnating the first-antibody spreading member with a certain amount of the first antibody and drying the first-antibody spreading member.

Particularly, glass fiber that typically has a weak adsorption power to the first antibody is preferable as the first-antibody spreading member, because there is a need that the first-antibody spreading member easily elute the first antibody when permeated by the testing target liquid in order for the first antibody to move together with the testing target liquid. For example, as the first-antibody spreading member, glass fiber or the like that supports the first antibody is disposed at such a position on the flow path member as upstream from the second-antibody immobilized portion in a state that the first-antibody spreading member can spread the first antibody. In this way, it is possible to perform testing only by dropping the testing target liquid onto a dropping portion described below, because this makes the target substance if any in the testing target liquid be bound with the first antibody by the first-antibody spreading member of the first-antibody supplying portion and then makes the target substance spread together with the first antibody toward the second-antibody immobilized portion. This can simplify the operation of the target testing device.

### <<First Antibody>>

The first antibody is not particularly limited, and an arbitrary first antibody may be selected according to the purpose so long as the first antibody can bind with the target substance. However, a labeled antibody containing a label is preferable. Examples of such a labeled antibody include a gold-colloid-labeled antibody such as gold-colloid-labeled anti-human IgG, and labeled antibodies against various allergens. Particles with which the antibody is labeled are not particularly limited to a gold colloid, and arbitrary particles may be selected according to the purpose. Examples of such particles include a metallic colloid, an enzymatic labeling particles containing an enzyme, coloring particles containing a pigment, fluorescent particles containing a fluorescent substance, and magnetic body-containing particles containing a magnetic body.

### <Second-Antibody Immobilized Portion>

The second-antibody immobilized portion is not particularly limited, and an arbitrary second-antibody immobilized portion may be selected according to the purpose so long as the second-antibody immobilized portion is a resin shaped body disposed at such a position on the flow path as downstream from the first-antibody supplying portion in a manner to contact the flow path, and so long as the second antibody bindable with at least one of the first antibody and the target substance is immobilized to such a surface of the shaped body as contacting the flow path by covalent binding with the shaped body. A plurality of second-antibody immobilized portions may be disposed over the flow path member, and different antibodies may be immobilized to the plurality of second-antibody immobilized portions, respectively.

### <<Shaped Body>>

The shaped body is not particularly limited, and an arbitrary shaped body may be selected according to the purpose so long as the shaped body is a resin shaped body. It is preferable that the second antibody be immobilized to such a surface of the shaped body as facing the flow path member.

Immobilizing the second antibody to the surface of the shaped body facing the flow path member makes it possible to control the strength of the covalent binding between the shaped body and the second antibody and affinity with the testing target liquid. Examples of a method for adjusting the strength of the covalent binding and the affinity include a method for varying the kind of the constituent resin of the shaped body and the composition ratio of the resin depending on the corresponding second antibody.

### <<<Resin>>>

The constituent resin of the shaped body is not particularly limited, and an arbitrary resin may be selected according to the purpose so long as the resin contains a functional group having bindability with the second antibody. However, a water-insoluble resin is preferable. The water-insoluble resin can avoid clogging the flow path or smudging a judgment line such as a control line or a test line without dissolving in the testing target liquid.

Examples of the functional group having bindability with the second antibody include a carboxyl group, an acid anhydride, an active ester group, an aldehyde group, an isocyanato group, an isothiocyanato group, a tosyl group, a pyridyl disulfide group, a bromo acetyl group, a hydroxyl group, an amino group, an epoxy group, a thiol group, a maleimide group, a vinyl sulfone group, an aminooxy acetyl group, a diazo group, a carbodiimide group, a vinyl group, a nitro group, a sulfone group, a succinimide group, a hydrazide group, an azido group, a phosphate group, an azlactone group, a nitrile group, an amide group, an imino group, a nitrene group, an acetyl group.

The active ester group refers to an ester group having a high reactivity. Specific examples of the active ester group include a p-nitrophenyl ester group, an N-hydroxyl succinimide ester group, a succinimide ester group, a phthalic imide ester group, and a 5-norbornene-2,3-dicarboxyimide ester group.

Among these, a carboxyl group, an acid anhydride, an active ester group, an aldehyde group, an isocyanato group, an isothiocyanato group, a tosyl group, a pyridyl disulfide group, a bromo acetyl group, a hydroxyl group, an amino group, an epoxy group, and a thiol group are preferable, and a carboxyl group, an amino group, and an active ester group are particularly preferable.

The shaped body needs only to contain the functional group over at least such a surface of the shaped body as facing the flow path member. The shaped body to which the functional group is incorporated by a known surface treatment method may be used. In this case, examples of the resin include a thermoplastic resin and a thermosetting resin. Of these, the thermoplastic resin is preferable in terms of production efficiency.

Examples of the thermoplastic resin include straight-chain polyolefins and cyclic polyolefins such as a polystyrene (PS) resin, a polyethylene (PE) resin, and a polypropylene (PP) resin, an ethylene vinyl acetate (EVA) copolymerized resin, an acrylonitrile styrene (AS) copolymerized resin, a methyl methacrylate (acrylic) (PMMA) resin, a polyamide (PA) resin, a polycarbonate (PC) resin, a polyethylene terephthalate (PET) resin, a polybutylene terephthalate (PBT) resin, a cellulose acetate (CA) resin, and a cycloolefin-based (CO) resin.

Examples of a constituent compound of the shaped body other than the resin include: natural waxes such as a beeswax, a carnauba wax, a cetaceum, a Japan tallow, a candelilla wax, a rice bran wax, and a montan wax; synthetic waxes such as a paraffin wax, a microcrystalline wax, an oxide wax, ozokerite, ceresin, an ester wax, a polyethylene wax, and a polyethylene oxide wax; higher fatty acids such as a margaric acid, a lauric acid, a myristic acid, a palmitic acid, a stearic acid, a furoic acid, and a behenic acid; higher alcohols such as a stearic alcohol and a behenyl alcohol; esters such as a fatty acid ester of sorbitan; and amides such as a stearic amide and an oleic amide.

One of these may be used alone, or two or more of these may be used in combination. Among these, a polystyrene resin, a polyolefin resin, a carnauba wax, and a polyethylene wax are preferable in terms of the purpose of use.

The surface treatment method for the shaped body is not particularly limited, and an arbitrary surface treatment method may be selected according to the purpose. For example, for incorporating the carboxyl group or the hydroxyl group to the surface of the shaped body, methods such as a plasma treatment, a corona discharge treatment, a flame treatment, and an ultraviolet irradiation treatment may be employed. Among these, a plasma treatment and a corona discharge treatment under an oxygen atmosphere are preferable because of the treatments' high reaction efficiency. For incorporating the amino group to the surface of the shaped body, methods such as a plasma treatment and an aminoalkyl silane treatment under a nitrogen atmosphere may be employed. Of these, the plasma treatment under the nitrogen atmosphere is preferable in terms of simplicity and uniformity of the treatment.

A non-porous body is preferable as the constituent resin of the shaped body. The non-porous body refers to a non-porous structure substantially free of voids, and a structure opposite to a porous material such as a membrane that contains voids provided for promoting absorption of a liquid. Hence, a material that contains only few cells that have been incidentally mixed in the material during a production process and that do not contribute to promotion of the liquid absorbing action is encompassed within the non-porous body.

Next, characteristics of the shaped body used in the present invention when the shaped body is a non-porous body will be described.

Conventional test line and control line are formed by directly applying a liquid in which the second antibody such as a capture antibody is dissolved over the flow path member made of a hydrophilic porous material. Hence, the second antibody is diffused inside the porous material along with permeation of the liquid. However, a color developed by labeling particles such as gold colloid particles bound with the second antibody present in the porous material cannot actually be sensed due to light scattering. This means that most of the second antibody is not used effectively.

Generally, color developing particles that can be sensed from the porous material are those that are present at and above the depth of about 5 μm from the surface of the porous material. In order to immobilize the second antibody needed for testing to the region at and above the depth of 5 μm, there is a need of applying the second antibody in a large amount considering diffusion of the second antibody in the direction of thickness. That is, the amount of the second antibody to be applied increases in proportion to the thickness of the porous material.

Meanwhile, in the present invention, when a resin shaped body made of a non-porous body containing many hydrophobic groups is used for immobilizing the second antibody, the second antibody is immobilized to only the surface of the shaped body without entering the inside of the resin shaped body. A color is developed when labeling particles bind with the second antibody immobilized to the surface of the shaped body. The color can be sensed through the shaped body made of the non-porous body that does not scatter light. This significantly improves the efficiency of utilization of the color developed by the labeling particles. Because there are no wasteful color developing particles in the direction of thickness, there is an advantage that the amount of the second antibody applied can be significantly saved. For example, when it is assumed that the thickness of the flow path member made of the hydrophilic porous material is 100 μm and color development from a region at and above the depth of 5 μm from the surface of the flow path member can only be utilized, the amount of the second antibody used for obtaining color development of the same intensity can be reduced to 1/20 in the present invention.

Hence, in the present invention, when the shaped body made of a non-porous body containing many hydrophobic groups is used for immobilizing the second antibody, the efficiency of utilization of the color developed by the labeling particles can be improved significantly, and the amount of the second antibody such as a capture antibody applied can be reduced from the amount used in conventional devices because there are no wasteful color developing particles in the direction of thickness.

In the present embodiment, it is preferable that the shaped body be immobilized to over the flow path member. A method for immobilizing the shaped body is not particularly limited so long as the shaped body can be immobilized in a state that enables the second antibody and the testing target liquid to contact each other during testing. Examples of the method include a method for thermally transferring the constituent resin of the shaped body onto the flow path member with a thermal transfer printer or the like, a method for applying a pressure to the constituent resin of the shaped body and transferring the resin with a dot impact printer or the like, and a method for pasting the constituent resin of the shaped body to the flow path member with a tape, an adhesive, a tackifier, etc.

### <<Second Antibody>>

The second antibody is not particularly limited, and an arbitrary second antibody may be selected according to the purpose so long as the second antibody contains a functional group covalently bindable with the functional group present over the surface of the shaped body and is bindable with at least one of the target substance and the first antibody. Examples of the second antibody include anti-human IgG, antibodies against various allergens, and an antibody such as human IgG against the first antibody. Besides, the second antibody may be in the form of a monoclonal antibody, a polyclonal antibody, a chimeric antibody, a Fab antibody, and a (Fab)2 antibody.

The functional group of the second antibody covalently bindable with the functional group present over the surface of the shaped body is not particularly limited, and an arbitrary functional group may be selected according to the purpose. Examples of the functional group include an amino group and a carboxyl group at a terminal of the second antibody, and an amino group, a carboxyl group, a thiol group, and a hydroxyl group at a side chain of the second antibody. A functional group may be newly incorporated into the second antibody for improving reactivity with the functional group of the shaped body. The position to which the new functional group is incorporated is not particularly limited, and may be appropriately selected according to the purpose. The position of incorporation may be a terminal of the molecular chain of the second antibody or a side chain of the second antibody when the functional group to be incorporated into the second antibody is an amino group. However, it is preferable to incorporate the amino group at the terminal of the molecular chain in order to immobilize the second antibody while preserving the inherent function of the physiological active substance.

A method for immobilizing the second antibody to the shaped body is not particularly limited, and an arbitrary method may be selected according to the purpose. Examples of the method include a method for forming covalent binding directly between the functional group present over the surface of the shaped body and the functional group contained in the second antibody, and a method for incorporating a compound having an appropriate chain length between the shaped body and the second antibody as a medium (spacer).

When using the shaped body containing the carboxyl group over the surface, it is possible to bind the second antibody to the shaped body by reacting an amino group in the molecule of the second antibody with the carboxyl group present over the surface of the shaped body in the presence of a dehydration condensation agent such as a water-soluble carbodiimide such as 1- ethyl-3-(3-dimethylaminopropyl)carbodiimide (EDC) to form amide binding. For further improving the reactivity, it is possible to further add N-hydroxysuccinimide (NHS) to the EDC to transform the carboxyl group to a reactive NHS ester and react the transformed carboxyl group with the amino group. When using the shaped body containing an aldehyde group over the surface, it is possible to use a method for reacting an amino group in the molecule of the second antibody with the aldehyde group to form a Schiff base and reacting a reducing agent such as hydrogenated sodium cyanoborate with the Schiff base to form a stable covalent binding.

As a specific operation for immobilizing the second antibody to the surface of the shaped body, a method for applying a liquid in which the second antibody is dissolved or dispersed is preferable. The pH of the liquid in which the second antibody is dissolved or dispersed is preferably in the range of from 6 through 10, and more preferably in the range of from 6 through 8. When the pH is lower than the lower limit, the efficiency of the immobilizing reaction is remarkably low. When the pH is higher than the upper limit, there is a high possibility that the second antibody will be denatured. However, the pH is not flatly limited and may be outside the range depending on the kind of the second antibody. is After the antibody is immobilized, it is possible to remove unnecessary components from the surface to which the antibody is immobilized by washing the surface with water containing a surfactant or a buffer fluid. When any functional group reactive with the second antibody remains over the surface to which the second antibody is immobilized, it is preferable to deactivate the functional group remaining over the surface with an alkali compound or a compound containing a primary amino group.

### <<<Covalent Binding>>>

The kind of the covalent binding is not particularly limited, and may be appropriately selected according to the purpose. However, amide binding, ester binding, thiourea bond, thioether bond, imine binding, and disulfide binding are preferable. Imine binding refers to R1−CH=N−R2, where R1 and R2 represent different alkyl groups. R1 and R2 may be the same. Particularly, in order to form covalent binding while substantially preserving the inherent physiological activity of the second antibody, a reaction in a liquid is indispensable, and there is a need of satisfying a condition that the pH of the reaction solution be in the range of from neutral through mild alkaline levels, and a condition that the reaction proceed in a short time at a reaction temperature in a range of from an ice cooling temperature through about 37° C. From these viewpoints, amide binding, thioether bond, and imine binding are preferable, and amide binding is particularly preferable. The inherent physiological activity refers to an antigen recognizing ability.

### <Other Members>

Other members are not particularly limited, and arbitrary members may be selected according to the purpose. Examples of the other members include a base material, an absorbing member, and a dropping portion.

### <<Base Material>>

The base material is of any form and any material that are not particularly limited and are selected according to the purpose.

The form of the base material is not particularly limited, and may be appropriately selected according to the purpose. Examples of the form include a form in which the flow path member is laminated over the top surface of the base material.

The constituent material of the base material is not particularly limited, and may be appropriately selected according to the purpose. Examples of the constituent material include organic, inorganic, or metallic materials. It is preferable that at least one surface of the base material be coated with a hydrophobic resin, although this is not limiting. When the target testing device is used as a sensor chip, it is preferable to use a lightweight, flexible, and inexpensive synthetic resin as the base material. It is optional to select a highly durable material such as a plastic sheet as the base material. This improves the durability of the target testing device as a result.

Examples of the base material include base materials made of polyvinyl chloride, polyethylene terephthalate, polypropylene, polystyrene, polyvinyl acetate, polycarbonate, polyacetal, modified polyphenylether, polybutylene phthalate, and an ABS resin. Among these, a base material made of polyethylene terephthalate is particularly preferable because a base material made of polyethylene terephthalate is low-price and highly versatile.

The shape of the base material is not particularly limited, and may be appropriately selected according to the purpose. However, a sheet shape is preferable.

The average thickness of the base material has no particular limit, and may be appropriately selected according to the purpose. However, the average thickness is preferably 0.01 mm or greater but 0.5 mm or less. When the average thickness is 0.01 mm or greater, the base material has an adequate strength as a base material. When the average thickness is 0.5 mm or less, the base material is flexible and suitable as a sensor.

In the present embodiment, the average thickness may be an average of thicknesses measured with a micrometer at a total of 15 positions of a measuring target, namely 5 positions in the longer direction×3 positions in the width direction that are selected at approximately equal intervals.

### <<Absorbing Member>>

The absorbing member is not particularly limited, and a known material may be selected as the absorbing member so long as the known material absorbs the liquid in the testing target liquid. For example, when the liquid is water, examples of the absorbing member include paper, fiber such as fabric, a polymeric compound containing a carboxyl group or a salt of a carboxyl group, a partially cross-linked product of a polymeric compound containing a carboxyl group or a salt of a carboxyl group, and a partially-cross-linked product of polysaccharides.

### <<Dropping Portion>>

The dropping portion is not particularly limited, and an arbitrary dropping portion may be selected according to the purpose so long as the dropping portion is formed at a place onto which the testing target liquid is dropped and is capable of supplying the testing target liquid to the flow path. The dropping portion may be selected from known materials.

Next, the target testing device of the present invention will be described in detail with reference to the drawings. In the present embodiment, two second-antibody immobilized portions are used, and different antibodies are immobilized to the two second-antibody immobilized portions. Hence, one second-antibody immobilized portion is referred to as “first second-antibody immobilized portion 50a”, and the other second-antibody immobilized portion is referred to as “second second-antibody immobilized portion 50b”.

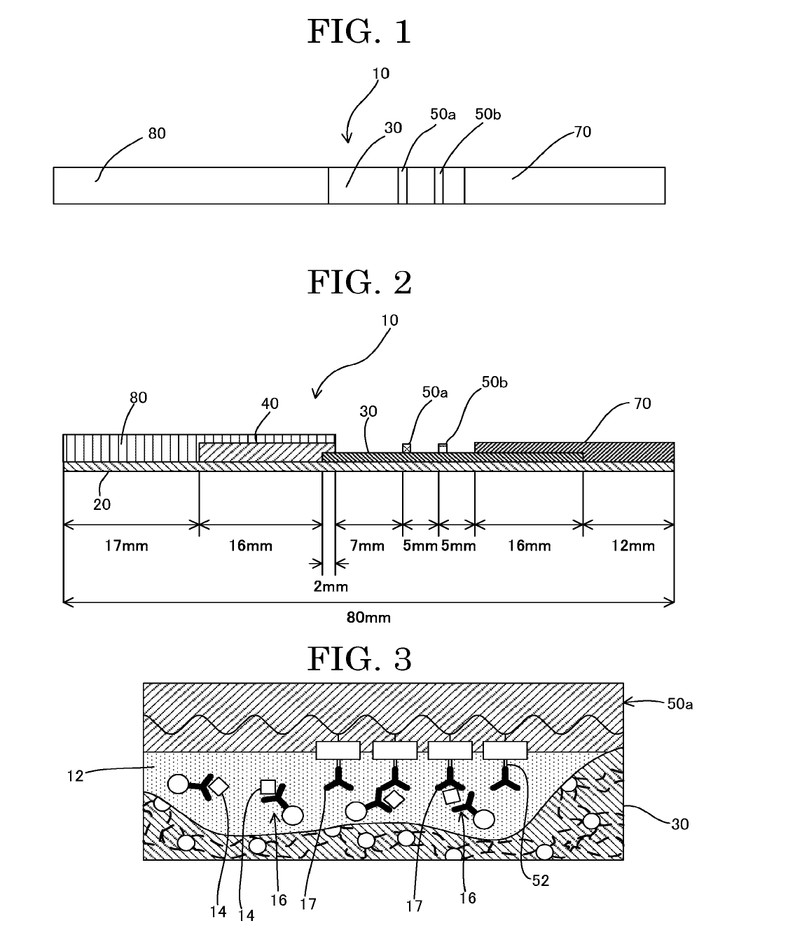

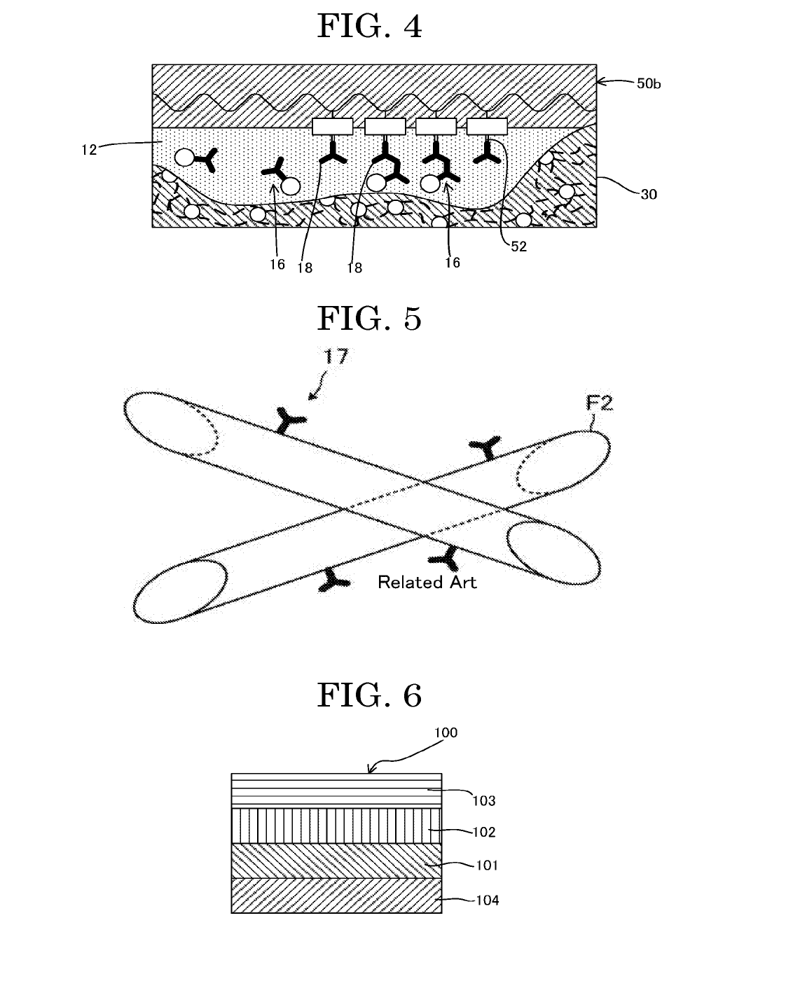

FIG. 1 is a top view of the target testing device according to an embodiment of the present invention. FIG. 2 is a cross-sectional view of the target testing device according to an embodiment of the present invention, the view being parallel with the longer direction of the target testing device. FIG. 3 is a partial enlarged view depicting a portion at which the first second-antibody immobilized portion and the flow path member contact each other. FIG. 4 is a partial enlarged view depicting a portion at which the second second-antibody immobilized portion and the flow path member contact each other.

As illustrated in FIG. 1 and FIG. 2, the target testing device 10 of the present invention includes a porous flow path member 30 in which the flow path for flowing the testing target liquid that is hydrophilic such as blood, a spinal fluid, urine, or an extraction liquid for testing (e.g., a liquid containing an analyte collected with an analyte collecting unit such as a stick) is formed, and a first-antibody supplying portion 40, a first second-antibody immobilized portion 50a, and a second second-antibody immobilized portion 50b that are provided over the flow path member 30. As illustrated in FIG. 3 and FIG. 4, a first second-antibody 17 and a second second-antibody 18 that are reactive with at least one of a target substance 14 contained in the testing target liquid 12 and the first antibody are immobilized to such surfaces of the first second-antibody immobilized portion 50a and second second-antibody immobilized portion 50b as facing the flow path member 30. In the present embodiment, the first second-antibody 17 is bindable with the target substance 14, and the second second-antibody 18 is bindable with the first antibody 16. This enables the strength of covalent binding between the shaped body and the antibody to be adjusted at the plurality of second-antibody immobilized portions individually. This makes it easy to control immobilization of the antibody even when the flow path member 30 is optionally selected according to the purpose.

In the following description, a case where the testing target liquid is a hydrophilic liquid such as blood, a spinal fluid, urine, or an extraction liquid for testing (e.g., a liquid containing an analyte collected with an analyte collecting unit such as a stick) will be described.

In the present embodiment, a case where as illustrated in FIG. 1 and FIG. 2, the flow path member 30 is provided over a base material 20, and an absorbing member 70 is provided over the base material 20 and the flow path member 30 at one end of the base material 20 and the flow path member 30 in the target testing device 10 will be described. However, the target testing device 10 of the present invention is not limited to this embodiment. In the present embodiment, what is meant when it is said that something is provided over a member is that that something is provided to contact the member regardless of whether that something is above or below the member when the target testing device 10 is set in place. When an arbitrary second-antibody immobilized portion of the first second-antibody immobilized portion 50a and the second second-antibody immobilized portion 50b is to be referred to, the arbitrary second-antibody immobilized portion will be denoted as second-antibody immobilized portion 50. The second antibody needs only to be immobilized by covalent binding.

As illustrated in FIG. 1 to FIG. 4, in the present embodiment, the first second-antibody immobilized portion 50a is used as a test line for judging presence or absence of the target substance 14, and the second second-antibody immobilized portion 50b is used as a control line for indicating that the first antibody 16 has arrived.

As illustrated in FIG. 1 and FIG. 2, the first-antibody supplying portion 40 of the present embodiment is disposed to contact the flow path member 30. As described above, the first-antibody supplying portion 40 supports the first antibody 16 in a state capable of spreading the first antibody 16 at a position upstream from the second-antibody immobilized portion 50. As illustrated in FIG. 2, the first-antibody supplying portion 40 supports the first antibody 16 over such a surface of the first-antibody supplying portion 40 as facing the flow path member 30.

As illustrated in FIG. 1 and FIG. 2, the first second-antibody immobilized portion 50a is disposed to contact the flow path member 30.

As illustrated in FIG. 3, the first second-antibody immobilized portion 50a contains a functional group bindable with the first second-antibody 17 over the surface of the first second-antibody immobilized portion 50a. The first second-antibody 17 contains a functional group bindable with the functional group present over the surface of the first second-antibody immobilized portion 50a, and by this functional group, forms covalent binding 52 and is immobilized to the surface of the first second-antibody immobilized portion 50a facing the flow path member 30. When a clearance formed between the flow path member 30 and the facing surface of the first second-antibody immobilized portion 50a is filled with the testing target liquid 12, the first second-antibody 17 captures the target substance 14 that is in a state of being bound with the first antibody 16.

Hence, a color is developed because the target substance 14 and the first antibody 16 have been immobilized. Therefore, the first second-antibody immobilized portion 50a can be used as a test line for judging presence or absence of the target substance 14. Incidentally, for the purpose of preventing inhibition of binding between the target substance and the antibody, the constituent resin of the shaped body of the first second-antibody immobilized portion 50a is a water-insoluble resin. In the present embodiment, water insolubility refers to a substantial water insolubility. Here, a resin is referred to as being substantially water-insoluble when the resin has undergone a mass change in an amount of 1% by mass or lower when the resin has been immersed in a large amount of water at 25° C. for 24 hours and then been sufficiently dried by a method such as vacuum drying. The reason why such a resin is substantially water-insoluble is that the mass change in an amount of 1% by mass or lower may be attributed to mass reduction due to leaching of a by-product (e.g., a monomer component) contained in the resin product into the water.

As illustrated in FIG. 1 and FIG. 2, the second second-antibody immobilized portion 50b is disposed to contact the flow path member 30 at a position downstream from the first second-antibody immobilized portion 50a. As illustrated in FIG. 4, the second second-antibody immobilized portion 50b contains a functional group bindable with the second second-antibody 18 over the surface of the second second-antibody immobilized portion 50b. The second second-antibody 18 contains a functional group bindable with the functional group present over the surface of the second second-antibody immobilized portion 50b, and by this functional group, forms covalent binding 52 and is immobilized to the surface of the second second-antibody immobilized portion 50b facing the flow path member 30. When a clearance formed between the flow path member 30 and the facing surface of the second second-antibody immobilized portion 50b is filled with the testing target liquid 12, the second second-antibody 18 captures the first antibody 16. Hence, a color is developed because the first antibody 16 has been immobilized. Therefore, the second second-antibody immobilized portion 50b can be used as a control line for indicating that the first antibody 16 has arrived. Like the first second-antibody immobilized portion 50a, the constituent resin of the shaped body at the second second-antibody immobilized portion 50b is a water-insoluble resin for the purpose of preventing inhibition of binding between the first antibody and the second antibody.

As illustrated in FIG. 1 and FIG. 2, a chopping portion 80 is disposed at an upstream end of the base material 20 in a manner to cover the first antibody supplying section 40.

In contrast to the dropping portion 80, the absorbing member 70 is disposed over the base material 20 at a downstream end of the base material 20 in a manner to cover the flow path member 30.

The target testing device of the present invention is not limited to one that utilizes an antigen-antibody reaction. For example, the target testing device may be configured to test a specific component contained in the testing target liquid by using as a reagent, a reagent that changes hues in response to a structural change.

As can be understood from a conceptual diagram of a membrane of a conventional testing device illustrated in FIG. 5, the second antibody 17 is immobilized to fibers F2 constituting the membrane in the conventional testing device. Hence, the second antibody 17 that can be immobilized to the membrane is limited to antibodies having a strong bindability with the fibers F2. That is, the conventional testing device is limited in design in terms of the fibers F2 and the second antibody 17 that can be used. In this regard, in the target testing device of the present invention, the shaped body and the reagent such as the second antibody are immobilized by covalent binding at the second-antibody immobilized portions. This is advantageous because it is possible to control the strength of covalent binding between the shaped body and the second antibody and affinity with the testing target liquid.

### (Transfer Medium)

A transfer medium of the present invention is a transfer medium for producing a target testing device, intended for producing the target testing device of the present invention. The transfer medium is not particularly limited, and an arbitrary transfer medium may be selected according to the purpose so long as the transfer medium is capable of forming the second-antibody immobilized portion by being transferred onto the flow path. The transfer medium includes a support member, a release layer, and a reagent immobilized layer, and may include other layers as needed.

### <Support Member>

The support member may be of any shape, any form, any size, and any material that are not particularly limited and may be appropriately selected according to the purpose.

The form of the support member is not particularly limited, and may be appropriately selected according to the purpose. Examples of the form include a single-layer form and a layered form.

The size of the support member is not particularly limited, and may be appropriately selected according to the size, etc. of the target testing device.

The material of the support member is not particularly limited, and may be appropriately selected according to the purpose. Examples of the material include polyesters such as polyethylene terephthalate (PET) and polyethylene naphthalate (PEN), polycarbonate, a polyimide resin (PI), polyamide, polyethylene, polypropylene, polyvinyl chloride, polyvinylidene chloride, polystyrene, a styrene-acrylonitrile copolymer, and cellulose acetate. One of these may be used alone, or two or more of these may be used in combination. Among these, polyethylene terephthalate (PET) and polyethylene naphthalate (PEN) are particularly preferable.

It is preferable to apply a surface activating treatment to the surface of the support member for improving close adhesiveness with the layer to be provided over the support member. Examples of the surface activating treatment include a glow discharge treatment and a corona discharge treatment.

The support member may be kept after the reagent immobilized layer described below is transferred onto the base material or the flow path member, or may be peeled off and removed by means of the release layer described below after the reagent immobilized layer is transferred.

The support member is not particularly limited, and may be an appropriately synthesized product or a commercially available product.

The average thickness of the support member has no particular limit, and may be appropriately selected according to the purpose. However, the average thickness is preferably 3 μm or greater but 50 μm or less.

### <Release Layer>

The release layer has a function of improving releasability between the support member and the reagent immobilized layer during transfer. The release layer also has a function of thermally fusing to become a low-viscosity liquid when heated with a heating pressurizing unit such as a thermal head and facilitating separation of the reagent immobilized layer at about the interface between the heated portion and a non-heated portion. The release layer contains a wax and a binder resin, and further contains other components appropriately selected as needed.

The wax is not particularly limited, and an arbitrary wax may be selected according to the purpose. Examples of the wax include: natural waxes such as a beeswax, a carnauba wax, a cetaceum, a Japan tallow, a candelilla wax, a rice bran wax, and a montan wax; synthetic waxes such as a paraffin wax, a microcrystalline wax, an oxide wax, ozokerite, ceresin, an ester wax, a polyethylene wax, and a polyethylene oxide wax; higher fatty acids such as a margaric acid, a lauric acid, a myristic acid, a palmitic acid, a stearic acid, a furoic acid, and a behenic acid; higher alcohols such as a stearic alcohol and a behenyl alcohol; esters such as a fatty acid ester of sorbitan; and amides such as a stearic amide and an oleic amide. One of these may be used alone, or two or more of these may be used in combination. Among these, a carnauba wax and a polyethylene wax are preferable because of excellent releasability.

The binder resin is not particularly limited, and an arbitrary binder resin may be selected according to the purpose. Examples of the binder resin include an ethylene-vinyl acetate copolymer, a partially saponified ethylene-vinyl acetate copolymer, an ethylene-vinyl alcohol copolymer, an ethylene-sodium methacrylate copolymer, polyamide, polyester, polyurethane, polyvinyl alcohol, methyl cellulose, carboxymethyl cellulose, starch, a polyacrylic acid, an isobutylene-maleic acid copolymer, a styrene-maleic acid copolymer, polyacrylamide, polyvinyl acetal, polyvinyl chloride, polyvinylidene chloride, an isoprene rubber, a styrene-butadiene copolymer, an ethylene-propylene copolymer, a butyl rubber, and an acrylonitrile-butadiene copolymer. One of these may be used alone, or two or more of these may be used in combination.

A method for forming the release layer is not particularly limited, and an arbitrary method may be selected according to the purpose. Examples of the method include a hot-melt coating method and a method for applying a coating liquid obtained by dispersing the wax and the binder resin in a solvent.

The average thickness of the release layer has no particular limit, and may be appropriately selected according to the purpose. However, the average thickness is preferably 0.5 μm or greater but 50 μm or less.

The amount of the release layer to be accumulated has no particular limit, and may be appropriately selected according to the purpose. However, the amount accumulated is preferably 0.5 g/m² or greater but 50 g/m² or less.

### <Reagent Immobilized Layer>

The reagent immobilized layer may be of any material that is not particularly limited and may be appropriately selected according to the purpose, so long as the reagent immobilized layer contains one of the constituent resins of the shaped bodies of the second-antibody immobilized portions of the target testing device.

A method for forming the reagent immobilized layer is not particularly limited, and an arbitrary method may be selected according to the purpose. Examples of the method include a hot-melt coating method and a method for applying a reagent-immobilized-layer coating liquid obtained by dispersing the constituent resin of the second-antibody immobilized portion in a solvent over the support member or the release layer by a common coating method such as a gravure coater, a wire bar coater, and a roll coater, and then drying the applied liquid.

The average thickness of the reagent immobilized layer has no particular limit, and may be appropriately selected according to the purpose. However, the average thickness is preferably 200 nm or greater but 50 μm or less. When the average thickness of the reagent immobilized layer is 200 nm or greater, the shaped body will have a good durability and will not be at a risk of being broken by rub or a shock. When the average thickness is 50 μm or less, heat from a thermal head is conducted uniformly through the reagent immobilized layer, and the shaped body is formed with a good sharpness.

The amount of the reagent coating liquid to be accumulated as the reagent immobilized layer has no particular limit, and may be appropriately selected according to the purpose. However, the amount accumulated is preferably 0.2 g/m² or greater but 50 g/m² or less. When the amount accumulated is 0.2 g/m² or greater, the amount of the reagent coating liquid applied is sufficient and the second-antibody immobilized portion will not have any lacking portion. When the amount accumulated is 50 g/m2 or less, drying will not take time, and the second-antibody immobilized portion will not have unevenness.

After the reagent coating liquid is dried and the reagent immobilized layer is formed, a liquid in which the second antibody is dissolved or dispersed is applied over the surface of the reagent immobilized layer to form a uniform coating film. After the second antibody is immobilized, the surface to which the second antibody is immobilized is washed with water containing a surfactant or a buffer fluid to remove unnecessary components. When any functional group reactive with the second antibody remains over the surface to which the second antibody is immobilized, it is preferable to deactivate the functional group remaining over the surface with an alkali compound or a compound containing a primary amino group. In the way described above, the second antibody can be immobilized to the surface of the reagent immobilized layer. It is preferable to apply the coating film to have a uniform thickness. Examples of a drying method include, but are not limited to through-flow drying, vacuum drying, natural drying, and freeze drying. Drying at a reduced pressure or in a vacuum is preferable. It is preferable to perform drying at a drying temperature that is selected from room temperature in a range of from 20° C. through 50° C. for a drying time in a range of from 30 minutes through 24 hours. When the drying temperature is higher than 20° C., the time taken for drying is saved and productivity is improved as a result. When the drying temperature is lower than 50° C., there is no risk of the reagent being denatured by heat.

When the drying time is longer than 30 minutes, drying is sufficient. When the drying time is shorter than 24 hours, productivity is improved and there is no need of considering discoloration of the resin.

### <Other Layers>

The other layers are not particularly limited, and arbitrary layers may be selected according to the purpose. Examples of the other layers include a back layer, an undercoat layer, and a protective film.

### <<Back Layer>>

The back layer is not particularly limited, and an arbitrary back layer may be selected according to the purpose. It is preferable that the transfer medium be provided with the back layer over such a surface of the support member as opposite to a surface of the support member over which the release layer is provided. During transfer, heat is directly applied to the opposite surface by a thermal head or the like in a manner to conform to the shape of the shaped body of the second-antibody immobilized portion. Hence, it is preferable that the back layer have resistance to a high heat and resistance to rub with a thermal head or the like. The back layer contains a binder resin, and further contains other components as needed.

The binder resin is not particularly limited, and an arbitrary binder resin may be selected according to the purpose. Examples of the binder resin include a silicone-modified urethane resin, a silicone-modified acrylic resin, a silicone resin, a silicone rubber, a fluororesin, a polyimide resin, an epoxy resin, a phenol resin, a melamine resin, and nitrocellulose. One of these may be used alone, or two or more of these may be used in combination.

The other components are not particularly limited, and arbitrary components may be selected according to the purpose. Examples of the other components include inorganic particles of talc, silica, organopolysiloxane, etc., and a lubricant.

A method for forming the back layer is not particularly limited, and an arbitrary method may be selected according to the purpose. Examples of the method include common coating methods such as a gravure coater, a wire bar coater, and a roll coater.

The average thickness of the back layer has no particular limit, and may be appropriately selected according to the purpose. However, the average thickness is preferably 0.01 μm or greater but 1.0 μm or less.

### <<Undercoat Layer>>

The undercoat layer may be provided between the support member and the release layer, or between the release layer and the reagent immobilized layer. The undercoat layer contains a resin, and further contains other components as needed. The resin is not particularly limited, and an arbitrary resin may be selected according to the purpose. The various resins used in the reagent immobilized layer and the release layer may be used.

### <<Protective Film>>

It is preferable to provide a protective film over the reagent immobilized layer in order to protect the reagent immobilized layer from contamination or damages during storage. The material of the protective film is not particularly limited, and an arbitrary material may be selected according to the purpose so long as the material can be easily peeled from the reagent immobilized layer. Examples of the material include silicone paper, polyolefin sheets such as polypropylene, and a polytetrafluoroethylene sheet. The average thickness of the protective film has no particular limit, and may be appropriately selected according to the purpose. However, the average thickness is preferably 5 μm or greater but 100 μm or less, and more preferably 10 μm or greater but 30 μm or less.

Conventionally, when a resin shaped body to which the second antibody is immobilized is manufactured into the form of a transfer medium for producing a testing device, there is a need of storing the transfer medium by winding the transfer medium around a core into a roll form in a multi-laminated state. If the force of binding between the second antibody and the resin shaped body is weak such as when the second antibody is physisorbed to the resin shaped body, it is likely that the reagent will come off to the back of the transfer medium (the back being the side opposite to the surface over which the reagent is formed in a solid phase). This raises a problem that the storage stability is low.

Meanwhile, in the present invention, the second antibody is immobilized by covalent binding, and this makes it harder for the reagent to come off to the back of the transfer medium even when the transfer medium is laminated. This provides an advantage that the storage stability is high.

### (Method for Producing Target Testing Device)

A method of the present invention for producing a target testing device includes a step of bringing the reagent immobilized layer of the transfer medium and the flow path member into contact with each other to transfer the reagent immobilized layer onto the flow path member, and further includes other steps as needed.

The step of transferring the reagent immobilized layer onto the flow path member is not particularly limited, and an arbitrary step may be selected according to the purpose. Examples of the step include a step based on a method for thermally transferring the reagent immobilized layer onto the flow path member.

Transferring of the reagent immobilized layer, which is the method for producing a target testing device, will be described in detail with reference to a drawing. FIG. 6 is a cross-sectional view illustrating a layered state of a transfer medium used for a target testing device.

Transferring of the reagent immobilized layer is not particularly limited by the structure of the flow path, etc. so long as the reagent immobilized layer is transferred onto the flow path. Hence, the reagent immobilized layer may be formed in an arbitrary manner according to the purpose. Here, a method for thermally transferring the reagent immobilized layer onto the flow path member will be described.

As illustrated in FIG. 6, a transfer medium 100 includes a support member 101, a release layer 102, and a reagent immobilized layer 103 in a layered state in the order of reciting, and further includes a back layer 104 over a surface of the support member 101 opposite to the surface over which the release layer 102 is layered.

Examples of the method for thermally transferring the reagent immobilized layer 103 onto the flow path member 30 include a method including a step of bringing the reagent immobilized layer 103 of the transfer medium 100 and the flow path member 30 into contact with each other to transfer the reagent immobilized layer 103 onto the flow path member 30. A printer used for thermal transfer is not particularly limited, and an arbitrary printer may be selected according to the purpose. Examples of the printer include thermal printers including a serial thermal head, a line thermal head, etc. The energy applied for thermal transfer has no particular limit, and may be appropriately selected according to the purpose. However, the energy is preferably 0.05 mJ/dot or higher but 0.5 mJ/dot or lower. When the applied energy is 0.05 mJ/dot or higher, the reagent immobilized layer 103 is fused sufficiently. When the applied energy is 0.5 mJ/dot or lower, there is no risk of the reagent being denatured by heat, and there is no risk of the other portions of the transfer medium 100 than the reagent immobilized layer 103 being fused, which prevents the thermal head from being contaminated.

### <<<Applications of Target Testing Device>>>

Applications of the target testing device are not particularly limited, and arbitrary applications may be selected according to the purpose. Examples of the applications include biochemical sensors (sensing chips) for blood testing and DNA testing, and small-size analytical devices (chemical sensors) for quality control of foods and beverages, etc.

Samples (analytes) used in biochemical testings are not particularly limited, and arbitrary samples may be selected according to the purpose. Examples of the samples include pathogens such as bacteria and viruses, and blood, saliva, lesional tissues, etc. separated from living organisms, or excretion such as enteruria. Further, for performing a prenatal diagnosis, the sample may be a part of a fetus cell in an amniotic fluid or a part of a dividing egg cell in a test tube. Furthermore, these samples may be, after condensed to a sediment is directly or by centrifugation or the like as needed, subjected to a pre-treatment for cell destruction through an enzymatic treatment, a thermal treatment, a surfactant treatment, an ultrasonic treatment, any combinations of these, etc.

The target testing device of the present embodiment also has a function of chromatographing (separating or refining) a testing target liquid because the flow path member functions as a static bed. In this case, the flow path member including the continuous cells of which internal wall has hydrophilicity functions as the static bed (or a support). Different components in the testing target liquid flow through the flow path at different speeds because of the difference in the interaction with the static bed during the process of permeating the flow path, i.e., the difference in whether the components are hydrophilic or hydrophobic.

A component having a higher hydrophilicity adsorbs to the porous portion functioning as the static bed more easily, and repeats adsorbing and desorbing more times, resulting in a lower permeating speed through the flow path. In contrast, a component having a higher hydrophobicity permeates the flow path without adsorbing to the static bed, and hence moves through the flow path more quickly. By extracting the target component in the testing target liquid selectively based on the difference in the moving speed in the testing target liquid and letting the target component undergo a reaction, it is possible to use the target testing device of the present invention as a highly functional chemical or biochemical sensor.

### (Target Testing Method)

A target testing method of the present invention is not particularly limited so long as the target testing method is a target testing method for performing testing with the target testing device of the present invention. The target testing method may include an analyte supplying step of supplying an analyte to the flow path member, and a step of capturing a part of the analyte by the second antibody immobilized to the shaped body.

In a specific operation, the testing target liquid 12 is dropped and supplied onto the dropping portion 80 (see FIG. 1 and FIG. 2) provided to the flow path member 30 of the target testing device 10. Next, the supplied testing target liquid 12 and the first antibody 16 supported over the first-antibody supplying portion 40 are brought into contact with each other, and the first antibody 16 is released from the first-antibody supplying portion 40. When any target substance 14 is contained in the testing target liquid 12, the first antibody 16 released from the first-antibody supplying portion 40 reacts and binds with the target substance 14.

Next, the testing target liquid 12 containing the first antibody 16 and the target substance 14 is spread along the flow path member 30, and arrives at the region at which the first second-antibody immobilized portion 50a is disposed. The first second-antibody 17 immobilized to the surface of the first second-antibody immobilized portion 50a facing the flow path member 30 binds with and captures the target substance 14 that is in the state of being bound with the first antibody 16. As described above, the first second-antibody 17 is immobilized to the shaped body by the covalent binding 52. Therefore, even when the first second-antibody 17 contacts the testing target liquid 12, the first second-antibody 17 does not become affinitive with the testing target liquid 12, and is not easily released into the testing target liquid 12. Even if some part of the first second-antibody 17 is released into the testing target liquid 12, the released part gets bound with the fibers constituting the flow path member 30 quickly. This facilitates immobilization of the first antibody 16 to about the first second-antibody immobilized portion 50a. As a result, the first second-antibody immobilized portion 50a functioning as the test line develops a color clearly.

In the present embodiment, any first antibody 16 that passes by the first second-antibody immobilized portion 50a without being captured is spread along the flow path member 30 and arrives at the region at which the second second-antibody immobilized portion 50b is disposed. At the second second-antibody immobilized portion 50b, the first antibody 16 is captured by being bound with the second second-antibody 18. Since the second second-antibody 18 is immobilized, the second second-antibody immobilized portion 50b functioning as the control line develops a color clearly.

### (Target Testing Kit)

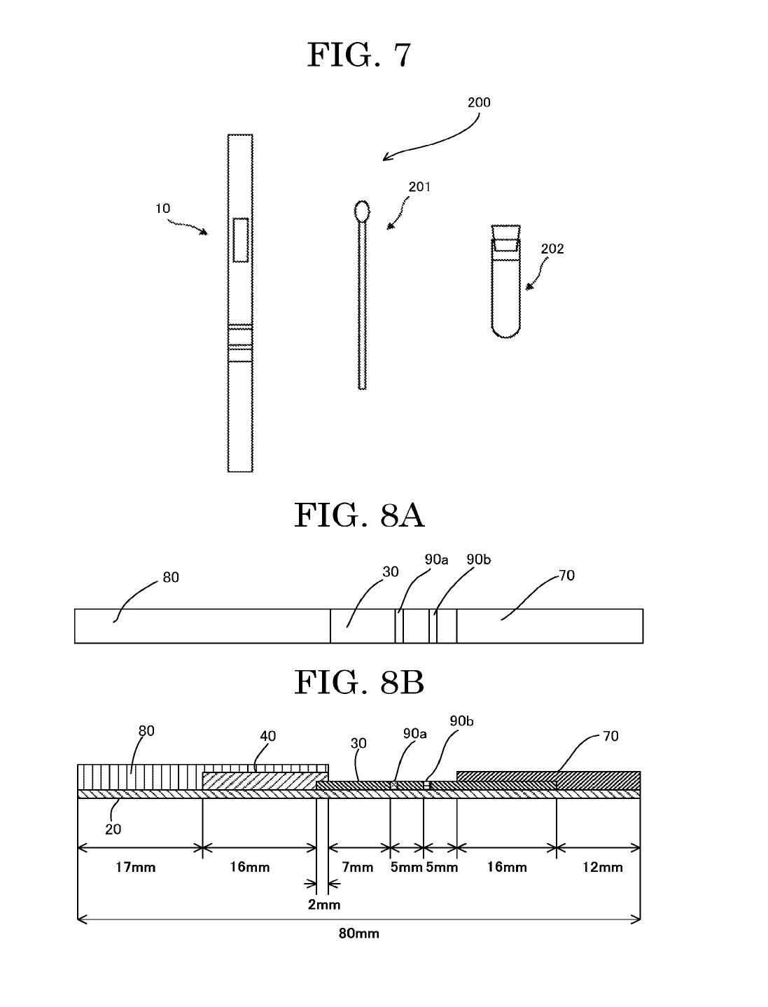

A target testing kit of the present invention is a target testing kit for performing testing with the target testing device of the present invention. For performing testing according to the target testing method described above, it is possible to use a target testing kit including the target testing device 10 and at least one of a tool for collecting an analyte (an example of the analyte collecting unit) and a liquid for treating an analyte as illustrated in FIG. 7. FIG. 7 is a conceptual diagram of a target testing kit 200 according to an embodiment of the present invention. Examples of the tool for collecting an analyte include known tools such as a sterilized cotton swab 201 for collecting an analyte from pharynx, nasal cavity, etc. Examples of the liquid for treating an analyte include known liquids such as a diluting fluid 202 for diluting an analyte, an extraction liquid for extracting an analyte, etc.

In the embodiment described above, a case where the reagents supplied from the first-antibody supplying portion and the second-antibody immobilized portions are an antigen or an antibody is described. The present invention is not limited to this embodiment. An indicator used in a chemical assay refers to a reagent for indicating a chemical property of a solution. The indicator is not particularly limited, and examples of the indicator include a pH indicator, various ionophores that discolor by reacting with various ions such as a lead ion, a copper ion, and a nitrite ion, and reagents that discolor by reacting with various agricultural chemicals.

In the embodiment described above, a case where the support member 101 and reagent immobilized layer 103 of the transfer medium 100 are separated from each other by heat during transfer is described. The present invention is not limited to this embodiment. For example, the support member 101 and the reagent immobilized layer 103 may be separated from each other by light. In this case, the release layer 102 may contain a light absorber such as carbon black and may make the light absorber absorb light and generate heat, so that the release layer 102 is fused and releases the reagent immobilized layer 103. Alternatively, the release layer 102 may contain a material that changes properties in response to light irradiation and may make the material absorb light, so that the release layer 102 is made fragile to release the reagent immobilized layer 103.

In the embodiment described above, a case where the flow path is formed throughout the flow path member 30 is described. The present invention is not limited to this embodiment. Examples of a method for forming a flow path in a partial region of the flow path member 30 include a method for forming a flow path wall defining an external edge of the flow path by filling the voids of the hydrophilic porous material with a hydrophobic material.

The testing device 10 of the present embodiment may be provided with an arbitrary protective member for preventing a hand from being contaminated by touching the flow path member 30. Examples of the protective member include a housing for entirely covering the testing device 10 and a film provided over the flow path member 30. When the protective member is provided, it is preferable to provide a hole at a position above the dropping portion 80 of the flow path member 30. It is also preferable to provide a hole in the protective member for dissipating the internal pressure in the flow path.

In the embodiment described above, a case where the testing target liquid is hydrophilic is described. The present invention is not limited to this embodiment. For example, the testing target liquid may be lipophilic or solvophilic. Examples of solvophilic testing target liquids include solvophilic liquids containing organic solvents such as alcohols such as methyl alcohol, ethyl alcohol, 1-propyl alcohol, and 2-propyl alcohol, and ketones such as acetone and methyl ethyl ketone (MEK). When the testing target liquid is solvophilic, the term “hydrophilic” in the embodiment described above is replaced by “hydrophobic”, and the term “hydrophobic” is replaced by “hydrophilic”.

### EXAMPLES

Examples of the present invention will be described below. The present invention is not limited to these Examples by any means.

### Example 1

### <Production of Thermal Transfer Media for Reagents>

### <<Preparation of Solutions for Transfer Media for Immobilization>>

### —Preparation of Back-Layer Coating Liquid—

A silicone-based rubber emulsion (KS779H available from Shin-Etsu Chemical Co., Ltd., with a solid component concentration of 30% by mass) (16.8 parts by mass), a chloroplatinic acid catalyst (0.2 parts by mass), and toluene (83 parts by mass) were mixed to obtain a back-layer coating liquid.

### —Preparation of Release Layer Coating Liquid—

A polyethylene wax (POLYWAX 1000 available from Toyo ADL Corporation, with a melting point of 99° C. and a penetration of 2 at 25° C.) (14 parts by mass), an ethylene-vinyl acetate copolymer (EV-150 available from Mitsui Du Pont Polychemicals Co., Ltd., with a weight average molecular weight of 2,100, and VAc of 21% by mass) (6 parts by mass), toluene (60 parts by mass), and methyl ethyl ketone (20 parts by mass) were subjected to a dispersion treatment until the average particle diameter became 2.5 μm to obtain a release layer coating liquid.

### <<Preparation of Reagent Coating Liquids>>

### —Test Line—

A PBS (Dulbecco's phosphate buffered saline D8662 available from Sigma-Aldrich Co. LLC) was added to an anti-human IgG antibody (I1886 available from Sigma-Aldrich Co. LLC) to be prepared to 10 μg/mL to obtain a test line reagent coating liquid. The anti-human IgG antibody had an amino group.

### —Control Line—

A PBS was added to human IgG (I2511-10MG available from Sigma-Aldrich Co. LLC) to be prepared to 10 μg/mL to obtain a control-line-reagent coating liquid. The human IgG had an amino group.

### —Labeled Antibody—

A labeled-antibody diluting fluid (a 20 mM tris-HCl buffer (pH=8.2), 0.05% by mass of polyethylene glycol, 5% by mass of sucrose, and purified water) was added to a gold-colloid-labeled anti-human IgG antibody to be prepared to OD=2 to obtain a labeled-antibody-reagent coating liquid.

### <Production of Transfer Media>

### <<Formation of Layers>>

### —Formation of Back Layer—

The back-layer coating liquid was applied over one surface of a support member which was a PET film having an average thickness of 4.5 μm (LUMIRROR F57 available from Toray Industries, Inc.), and dried at 80° C. for 10 seconds to form a back layer having an average thickness of 0.02 μm.

### —Formation of Release Layer—

Next, the release layer coating liquid was applied over a surface of the PET film opposite to the surface over which the back layer was formed, and dried at 25° C. for 30 minutes to form a release layer having an average thickness of 30 μm.

### —Hydrophilization of Release Layer Surface—

Next, the surface of the release layer was oxidized by a plasma treatment under an oxygen atmosphere to incorporate a hydrophilic group including a carboxyl group into the release layer and impart a function as the reagent immobilized layer to the release layer to produce a transfer medium.

### —Activation of Carboxyl Group—

1-Ethyl-3-(3- dimethyl aminopropyl)carbodiimide (WSC available from Dojindo Laboratories) was prepared with a PBS to 10 mg/mL and poured into a shallow square vat. Next, the vat was lidded in a state that the transfer medium was floated in the aqueous solution such that only the surface of the reagent immobilized layer contacted the WSC aqueous solution, and then stood still at 37° C. for 2 hours. After this, the surface of the reagent immobilized layer was washed with a PBS.

### <Immobilization of Second Antibodies>

### —Test Line (First Second-Antibody)—

The test-line-reagent coating liquid was poured into a shallow square vat. Then, the vat was lidded in a state that the transfer medium was floated in the aqueous solution such that only the surface of the reagent immobilized layer contacted the test-line-reagent coating liquid, and then stood still at 37° C. for 2 hours. After this, the surface of the reagent immobilized layer was washed with a PBS, dried in a vacuum at 25° C. for 30 minutes to immobilize the reagent to the reagent immobilized layer. In the way described above, a thermal transfer medium for a test line was obtained.

### —Control Line (Second Second-Antibody)—

A thermal transfer medium for a control line was obtained in the same manner as for obtaining the thermal transfer medium for a test line, except that the control-line-reagent coating liquid was used instead of the test-line-reagent coating liquid.

It was confirmed by FT-ATR that the first second-antibody and the second second-antibody were covalently bound with the reagent immobilized layer.

### <Production of Testing Devices>

### <<Production of Paper Substrates (Substrate+Flow Path Member)>>

As a thermoplastic resin, a polyester-based hot-melt adhesive (ARONMELT PES375S40 available from Toagosei Co., Ltd.) was heated to 190° C., and then with a roll coater, applied over a PET film (LUMIRROR S10 available from Toray Industries, Inc., with an average thickness of 50 μm) cut into a size of 40 mm in width and 80 mm in length to have a thickness of 50 μm over the PET film to form an adhesive layer. The applied product was stood still for 2 hours or longer. After this, each of the flow path members presented in Table 1 each cut into a size of 40 mm in width and 35 mm in length was overlapped with the adhesive layer-applied surface of the applied product at a position that was apart by 33 mm from one end of the adhesive layer-applied surface in the longer direction (the one end being an upstream end, the opposite end being a downstream end) in a state that the flow path member and the applied product coincided widthwise, and a load of 1 kgf/cm² was imposed to the overlapped product at a temperature of 150° C. for 10 seconds. Finally, the overlapped product was cut along the longer direction of the overlapped product into a size of 4 mm in width and 80 mm in length, to obtain a paper substrate.

The voidage of the flow path members A to E was calculated according to a calculation formula 1 below based on a basis weight (g/m²) and an average thickness (μm) of the paper substrate, and the specific gravity of the components of the paper substrate.

Voidage %={1−[basis weight (g/m²)/average thickness (μm)/specific gravity of the components]}×100 [Calculation formula 1]

When the voidage of the flow path members is 40% or higher but 90% or lower, the flow path members can be said to be porous. From the results presented in Table 1 below, all of the flow path members A to E were porous.

TABLE 1

| | Kind of flow path member | Voidage (%) |
|---|---|---|
| A | Nitrocellulose membrane filter (HF240 available | 70 |
| | from Merck Millipore Inc.) | |
| B | Hydrophilic PTFE (polytetrafluoroethylene) | 80 |
| | (JMWP14225 available from Merck Millipore Inc.) | |
| C | Hydrophilic PVDF (polyvinylidene fluoride) | 70 |
| | (SVLP04700 available from Merck Millipore Inc.) | |
| D | Qualitative filter (Qualitative filter No. 4A | 48 |
| | available from Advantec Co., Ltd.) | |
| E | Groundwood paper (TA-914 available from Maruai | 70 |
| | Inc.) | |

### <Transfer of Reagents>

### —Test Line and Control Line—

Each paper substrate and the reagent immobilized side of the thermal transfer medium for the reagent were faced and overlapped with each other, and then with a thermal transfer printer, the thermal transfer medium for the test line was transferred onto a position that was apart by 9 mm from the upstream end of the flow path member in a line shape having a width of 4 mm and a length of 0.7 mm, as illustrated in FIG. 1 and FIG. 2. Further, the thermal transfer medium for the control line was transferred onto a position that was apart by 5 mm from the thermal transfer medium for the test line in a line shape having a width of 4 mm and a length of 0.7 mm. For forming the patterns, an evaluation system having a printing speed of 42 mm/sec and a printing energy of 0.17 mJ/dot was constructed with a thermal head having a dot density of 300 dpi (available from TDK Corporation) for evaluation of the printing.

### —Labeled-Antibody Supporting Pad (First-Antibody Supplying Portion 40)—

The labeled-antibody-reagent coating liquid produced in the preparation of reagent coating liquids described above was applied in an amount of 60 μL/cm² over a glass-fiber pad (GFCP203000 available from Merck Millipore Inc.) cut into a size of 4 mm in width and 18 mm in length and dried overnight at a reduced pressure to produce a labeled-antibody supporting pad (first-antibody supplying portion 40). The obtained labeled-antibody supporting pad was placed at a position that was apart by 17 mm from the upstream end of the substrate as illustrated in FIG. 1 and FIG. 2.

### —Sample Dropping Pad (Dropping Portion 80)—

A sample pad having a size of 4 mm in width and 35 mm in length (CFSP22300 available from Merck Millipore Inc.) was overlapped with the top surface of the labeled-antibody supporting pad by 18 mm as illustrated in FIG. 1 and FIG. 2 and pasted at the position to form a sample dropping pad (dropping portion 80).

### —Absorbing Member—

Further, an absorbing member 70 (CFSP223000 available from Merck Millipore Inc.) was disposed as illustrated in FIG. 1 and FIG. 2.

In the way described above, immunochromatoassays (target testing devices 10) A to E were obtained.

### [1] Evaluation of Visibility of Lines

### <<Evaluation Method>>

### 1. Preparation of Testing Target Liquid

An antibody diluting fluid (Dulbecco's phosphate buffered saline D8662 available from Sigma-Aldrich Co. LLC) was added to human IgG to be prepared to 500 μg/mL to obtain a testing target liquid.

### 2. Reaction

The testing target liquid (100 μL) was dropped onto the upstream end portion of the immunochromatoassays A to E, and when 30 minutes passed, the immunochromatoassays were visually observed. Among the immunochromatoassays, those over which a clear color development was recognized at the positions of the test line and the control line at a uniform color optical density throughout the lines without discontinuation of the lines were evaluated as A, those over which the lines were not discontinuous and enabled judgment but were slightly non-uniform in the color optical density from place to place were evaluated as B, those over which color development was barely recognized as line shapes but with a partial discontinuation in the lines were evaluated as C, and those over which no color development was recognized or color development was not in line shapes such as when the lines flowed to the downstream side were evaluated as D. Examples of the evaluation criteria are presented in Table 2. The pictures in Table 2 are each a picture of the test line after testing. Evaluation results are presented in Table 3.

### [2] Measurement of Density of Lines

The immunochromatoassays after having developed colors used in [1] were housed in a housing case for measurement, and measured with a chromatoreader (DIASCAN 10 available from Otsuka Electronics Co., Ltd.) to obtain the optical density of the lines. A higher optical density is preferred. In the case of immunochromatoassays, those with an optical density of higher than or equal to 250 are evaluated as A, those with an optical density of lower than 250 but higher than or equal to 150 are evaluated as B, those with an optical density of lower than 150 but higher than or equal to 50 are evaluated as C, and those with an optical density of lower than 50 or those over which no lines are recognized and an optical density is unmeasurable are evaluated as D. In the case of chemical assays, those with an optical density of higher than or equal to 400 are evaluated as A, those with an optical density of lower than 400 but higher than or equal to 250 are evaluated as B, those with an optical density of lower than 250 but higher than or equal to 100 are evaluated as C, and those with an optical density of lower than 100 or those over which no lines are recognized and an optical density is unmeasurable are evaluated as D. Evaluation results are presented in Table 4.

### Example 2

### <Production of Thermal Transfer Media for Reagents>

### <<Preparation of Solutions for Transfer Media for Immobilization>>

### —Preparation of Back-Layer Coating Liquid—

A silicone-based rubber emulsion (KS779H available from Shin-Etsu Chemical Co., Ltd., with a solid component concentration of 30% by mass) (16.8 parts by mass), a chloroplatinic acid catalyst (0.2 parts by mass), and toluene (83 parts by mass) were mixed to obtain a back-layer coating liquid.

### —Preparation of Release Layer Coating Liquid—

A polyethylene wax (POLYWAX 1000 available from Toyo ADL Corporation, with a melting point of 99° C. and a penetration of 2 at 25° C.) (14 parts by mass), an ethylene-vinyl acetate copolymer (EV-150 available from Mitsui Du Pont Polychemicals Co., Ltd., with a weight average molecular weight of 2,100, and VAc of 21% by mass) (6 parts by mass), toluene (60 parts by mass), and methyl ethyl ketone (20 parts by mass) were subjected to a dispersion treatment until the average particle diameter became 2.5 μm to obtain a release layer coating liquid.

### —Preparation of Reagent-Immobilized-Layer Coating Liquid—

A mixture solution of toluene/methyl isobutyl ketone (MIBK) (3/1 on a mass ratio) was added to an aminoethylated acrylic polymer (POLYMENT NK-380 available from Nippon Shokubai Co., Ltd.) to dilute the aminoethylated acrylic polymer to 10% by mass to obtain a reagent-immobilized-layer coating liquid.

### <Production of Transfer Media>

### <<Formation of Layers>>

### —Formation of Back Layer—

The back-layer coating liquid was applied over one surface of a support member which was a PET film having an average thickness of 4.5 (LUMIRROR F57 available from Toray Industries, Inc.), and dried at 80° C. for 10 seconds to form a back layer having an average thickness of 0.02 μm.

### —Formation of Release Layer—

Next, the release layer coating liquid was applied over a surface of the PET film opposite to the surface over which the back layer was formed, and dried at 25° C. for 30 minutes to form a release layer having an average thickness of 30 μm.

### —Formation of Reagent Immobilized Layer—

Further, the reagent-immobilized-layer coating liquid was applied over the surface over which the release layer was formed, and dried at 50° C. for 30 minutes to form a reagent immobilized layer having an average thickness of 3 μm to obtain a transfer medium.

### —Activation of Amino Group—

A glutaraldehyde aqueous solution (G5882 available from Sigma-Aldrich Co. LLC) was diluted with a PBS to 1% by mass and poured into a shallow square vat. Next, the vat was lidded in a state that the transfer medium was floated in the aqueous solution such that only the surface of the reagent immobilized layer contacted the glutaraldehyde aqueous solution, and then stood still at 37° C. for 2 hours. After this, the surface of the reagent immobilized layer was washed with purified water.

### <Immobilization of Second Antibodies>

### —Test Line (First Second-Antibody)—

The test-line-reagent coating liquid was poured into a shallow square vat. Then, the vat was lidded in a state that the transfer medium was floated in the aqueous solution such that only the surface of the reagent immobilized layer contacted the test-line-reagent coating liquid, and then stood still at 37° C. for 2 hours. After this, the surface of the reagent immobilized layer was washed with a PBS. Then, an aqueous solution obtained by diluting a skim milk (198-10605 available from Wako Pure Chemical Industries, Ltd.) with a PBS to 3% by mass was poured into a shallow square vat. Then, the vat was lidded in a state that the transfer medium was floated in the aqueous solution such that only the surface of the reagent immobilized layer contacted the aqueous solution, and then stood still at 37° C. for 2 hours. After this, the surface of the reagent immobilized layer was washed with a PBS and dried in a vacuum at 25° C. for 30 minutes to immobilize the reagent to the reagent immobilized layer. In the way described above, a thermal transfer medium for a test line was obtained.

### —Control Line (Second Second-Antibody)—

A thermal transfer medium for a control line was obtained in the same manner as for obtaining the thermal transfer medium for a test line, except that the control-line-reagent coating liquid was used instead of the test-line-reagent coating liquid.

### <Production of Testing Devices>

Immunochromatoassays of Example 2 were produced in the same manner as in Example 1 except that the thermal transfer medium for a test line and the thermal transfer medium for a control line produced in Example 2 were used. The produced immunochromatoassays were evaluated.

### Example 3

### <Production of Thermal Transfer Media for Reagents>

### <<Preparation of Solutions for Transfer Media for Immobilization>>

### —Preparation of Back-Layer Coating Liquid—

A silicone-based rubber emulsion (KS779H available from Shin-Etsu Chemical Co., Ltd., with a solid component concentration of 30% by mass) (16.8 parts by mass), a chloroplatinic acid catalyst (0.2 parts by mass), and toluene (83 parts by mass) were mixed to obtain a back-layer coating liquid.

### Preparation of Release Layer Coating Liquid—

A polyethylene wax (POLYWAX 1000 available from Toyo ADL Corporation, with a melting point of 99° C. and a penetration of 2 at 25° C.) (14 parts by mass), an ethylene-vinyl acetate copolymer (EV-150 available from Mitsui Du Pont Polychemicals Co., Ltd., with a weight average molecular weight of 2,100, and VAc of 21% by mass) (6 parts by mass), toluene (60 parts by mass), and methyl ethyl ketone (20 parts by mass) were subjected to a dispersion treatment until the average particle diameter became 2.5 μm to obtain a release layer coating liquid.

### —Preparation of Reagent-Immobilized-Layer Coating Liquid—

A mixture solution of toluene/methyl isobutyl ketone (MIBK) (3/1 on a mass ratio) was added to an aminoethylated acrylic polymer (POLYMENT NK-380 available from Nippon Shokubai Co., Ltd.) to dilute the aminoethylated acrylic polymer to 10% by mass to obtain a reagent-immobilized-layer coating liquid.

### <<Preparation of Reagent Coating Liquids>>

### —Test Line—

EDTA (ethylene diamine tetraacetic acid: E0084 available from Tokyo Chemical Industry Co., Ltd.) prepared to 1 mM with purified water and a 0.1 M PBS (D1408 available from Sigma-Aldrich Co. LLC) were mixed at a ratio of 1:1 to prepare an EDTA-PBS solution. Next, the EDTA-PBS solution was added to an anti-human IgG antibody to be prepared to 2 mg/mL. Then, 2-MEA.HCl (2-mercaptoethylamine hydrochloride: 21419-74 available from Nacalai Tesque, Inc.) (5 mg) was added to the prepared liquid (1 mL), which was then incubated at 37° C. for 90 minutes. After this, the resultant was gel-filtered through a column (SEPHADEX G-25 available from GE Healthcare Co., Ltd.) that was equilibrated with the EDTA-PBS to remove unreacted 2-MEA to obtain a test-line-reagent coating liquid.

### —Control Line—

Human IgG was treated in the same manner as for the test line to obtain a control-line-reagent coating liquid.

### <Production of Transfer Media>

### <<Formation of Layers>>

### —Formation of Back Layer—

The back-layer coating liquid was applied over one surface of a support member which was a PET film having an average thickness of 4.5 (LUMIRROR F57 available from Toray Industries, Inc.), and dried at 80° C. for 10 seconds to form a back layer having an average thickness of 0.02 μm.

### —Formation of Release Layer—

Next, the release layer coating liquid was applied over a surface of the PET film opposite to the surface over which the back layer was formed, and dried at 25° C. for 30 minutes to form a release layer having an average thickness of 30 μm.

### Formation of Reagent Immobilized Layer—

Further, the reagent-immobilized-layer coating liquid was applied over the surface over which the release layer was formed, and dried at 50° C. for 30 minutes to form a reagent immobilized layer having an average thickness of 3 μm to obtain a transfer medium.

### —Incorporation of Maleimide Group—

N-maleimidocaproyloxy succinimide (EMCS available from Dojindo Laboratories) was dissolved in a mixture solvent of dimethyl sulfoxide/ethanol (2/8 at a mass ratio) to prepare a 0.4 mg/mL EMCS solution, which was then poured into a shallow vat. Next, the vat was lidded in a state that the transfer medium was floated in the solution such that only the surface of the reagent immobilized layer contacted the EMCS solution, and then stood still at 25° C. for 2 hours. After this, the reagent immobilized layer was washed with the dimethyl sulfoxide/ethanol (2/8 at a mass ratio) mixture solution.

### <Immobilization of Second Antibodies>

### —Test Line (First Second-Antibody)—

The test-line-reagent coating liquid was poured into a shallow square vat. The vat was lidded in a state that the transfer medium was floated in the aqueous solution such that only the surface of the reagent immobilized layer contacted the test-line-reagent coating liquid, and then stood still at 25° C. for 2 hours. After this, the surface of the reagent immobilized layer was washed with a PBS and dried in a vacuum at 25° C. for 30 minutes to immobilize the reagent to the reagent immobilized layer. In the way described above, a thermal transfer medium for a test line was obtained.

### —Control Line (Second Second-Antibody)—

A thermal transfer medium for a control line was obtained in the same manner as for obtaining the thermal transfer medium for a test line, except that the control-line-reagent coating liquid was used instead of the test-line-reagent coating liquid.

### <Production of Testing Devices>

Immunochromatoassays of Example 3 were produced in the same manner as in Example 1 except that the thermal transfer medium for a test line and the thermal transfer medium for a control line produced in Example 3 were used. The produced immunochromatoassays were evaluated.

### Example 4

Immunochromatoassays (testing devices 10) of Example 4 were produced in the same manner as in Example 1, except that the anti-human IgG antibody used in the test-line-reagent coating liquid in Example 1 was changed to an anti-hCG monoclonal antibody (ANTI-ALPHA SUBUNIT 6601 SPR-5 available from Medix Biochemica Inc.), the human IgG (I2511-10MG available from Sigma-Aldrich Co. LLC) used in the control-line-reagent coating liquid in Example 1 was changed to an anti-mouse IgG antibody (566-70621 available from Wako Pure Chemical Industries, Ltd.), and the gold-colloid-labeled anti-human IgG antibody used in the labeled-antibody-reagent coating liquid in Example 1 was changed to a gold-colloid-labeled antibody produced in the manner described below. The anti-hCG monoclonal antibody and the anti-mouse IgG antibody had an amino group.

### <Production of Labeled-Antibody-Reagent Coating Liquid>

One milliliter of a KH₂PO₄ buffer (pH=7.0) prepared to 50 mM, and then 1 mL of an anti-hCG monoclonal antibody (ANTI-HCG 5008 SP-5 available from Medix Biochemica Inc.) prepared to 50 μg/mL were added to 9 mL of a gold-colloid solution (EMGC50 available from Boston Biomedical Inc.), which was then stirred. The resultant was stood still for 10 minutes. To the resultant, 550 A of a 1% by mass polyethylene glycol aqueous solution (168-11285 available from Wako Pure Chemical Industries, Ltd.) was added and stirred. To the resultant, 1.1 mL of a 10% by mass BSA aqueous solution (A-7906 available from Sigma-Aldrich Co. LLC) was further added and stirred.

Next, the resulting solution was centrifuged for 30 minutes, and the supernatant was removed from the solution except for about 1 mL of the supernatant. The solution with the remaining supernatant was subjected to a redispersion treatment using an ultrasonic washing machine for dispersing the gold colloid. The centrifugation was performed with a centrifuge (HIMAC CF16RN available from Hitachi Koki Co., Ltd.) at a centrifugal acceleration of 8,000×g and at 4° C. After this, the resultant was dispersed in 20 mL of a gold colloid preservative solution (a 20 mM tris-HCl buffer (pH=8.2), 0.05% polyethylene glycol, 150 mM NaCl, a 1% by mass BSA aqueous solution, and a 0.1% by mass NaN₃ aqueous solution). The resultant was centrifuged again under the same conditions as described above, and the supernatant was removed except for about 1 mL of the supernatant. The solution with the remaining supernatant was subjected to a redispersion treatment using an ultrasonic washing machine for dispersing the gold colloid, to obtain a gold-colloid-labeled antibody. After this, the gold-colloid-labeled antibody produced above was diluted with a gold-colloid coating liquid and purified water to OD=10, to obtain a labeled-antibody-reagent coating liquid.

### [1] Evaluation of Visibility of Lines

### <Evaluation Method>

### 1. Preparation of Testing Target Liquid

An antibody diluting fluid (Dulbecco's phosphate buffered saline D8662 available from Sigma-Aldrich Co. LLC) was added to hCG (recombinant hCG, 7727-CG-010 available from R&D Systems, Inc.) to be prepared to 50 mIU/mL to obtain a testing target liquid.

### 2. Reaction

The testing liquid (100 μL) was dropped onto the upstream end portion of the immunochromatoassays A to E, and when 30 minutes passed, the immunochromatoassays were observed and evaluated in the same manner as in Example 1. Evaluation results are presented in Table 3.

### [2] Measurement of Density of Lines

An optical density of lines were measured in the same manner as in Example 1. Evaluation results are presented in Table 4.

### Comparative Example 1

### <Preparation of Reagent Coating Liquids>

### —Test Line—

An antibody diluting fluid was added to an anti-human IgG antibody to be prepared to 0.9 mg/mL to obtain a test-line-reagent coating liquid.

### —Control Line—

An antibody diluting fluid was added to human IgG to be prepared to 0.9 mg/mL to obtain a control-line-reagent coating liquid.

### —Labeled Antibody—

A labeled-antibody diluting fluid was added to a gold-colloid-labeled anti-human IgG antibody to be prepared to OD=2 to obtain a labeled-antibody-reagent coating liquid.

### <Production of Testing Devices>

### <<Production of Paper Substrates>>

As a thermoplastic resin, a polyester-based hot-melt adhesive (ARONMELT PES375S40 available from Toagosei Co., Ltd.) was heated to 190° C., and then with a roll coater, applied over a PET film (LUMIRROR S10 available from Toray Industries, Inc., with an average thickness of 50 μm) cut into a size of 40 mm in width and 35 mm in length to have an average thickness of 50 μm over the PET film to form an adhesive layer. The applied product was stood still for 2 hours or longer. After this, each of the flow path members presented in Table 1 each cut into the same size as the PET film was overlapped with the adhesive layer-applied surface of the applied product, and a load of 1 kgf/cm² was imposed to the overlapped product at a temperature of 150° C. for 10 seconds to obtain a paper substrate.

### <<Immobilization of Reagents>>

With a positive-pressure spray device (BIOJET available from BioDot, Inc.), the test-line-reagent coating liquid was applied at a position that was apart by 9 mm from the upstream end of the flow path member 30 in a line shape having a length of 0.7 mm (test line 90a) as illustrated in FIG. 8A and FIG. 8B. FIG. 8A is a top view of a target testing device according to the comparative example. FIG. 8B is a cross-sectional view of the target testing device of FIG. 8A, the view being parallel with the longer direction of the target testing device. Then, with the positive-pressure spray device, the control-line-reagent coating liquid was further applied at a position that was apart by 5 mm from the test line 90a in a line shape having a length of 0.7 mm (control line 90b). After the application, the applied liquids were dried at 20° C. at 20RH % for 16 hours.

### <<Production of Labeled-Antibody Supporting Pad (First-Antibody Supplying Portion)>>

The produced labeled antibody solution was applied in an amount of 60 μL/cm² over a glass-fiber pad (GFCP203000 available from Merck Millipore Inc.) cut into a size of 40 mm in width and 18 mm in length, and dried at a reduced pressure overnight to produce a labeled-antibody supporting pad (first-antibody supplying portion) 40.

### <Assembling of Assays (Target Testing Devices)>

The flow path member 30 was bonded to a base film which was a PET film (LUMIRROR S10 available from Toray Industries, Inc., with an average thickness of 100 μm) cut into a size of 40 mm in width and 80 mm in length, at a position that was apart by 33 mm from one end of the base film (PET film) in the longer direction such that a surface of the flow path member opposite to the surface over which the reagents were applied was faced with the base film (PET film).

Next, the labeled-antibody supporting pad produced above that was cut into a size of 40 mm in width and 18 mm in length was disposed over the top surface of the flow path member 30 in a manner to overlap the upstream end of the flow path member 30 by 2 mm and pasted at the position. Further, a sample pad having a size of 40 mm in width and 35 mm in length (CFSP223000 available from Merck Millipore Inc.) was overlapped with the top surface of the labeled-antibody supporting pad by 18 mm and pasted at the position to obtain a sample dropping pad (dropping portion) 80.

Next, an absorbing pad having a size of 40 mm in width and 28 mm in length (CFSP223000 available from Merck Millipore Inc.) was disposed over the top surface of the flow path member 30 in a manner to overlap the downstream end of the flow path member 30 by 16 mm and pasted at the position to provide an absorbing member 70. Finally, the obtained product was cut along the longer direction of the product into a size of 4 mm in width and 80 mm in length to obtain immunochromatoassays (testing devices 10) A to E of Comparative Example 1.

The produced immunochromatoassays A to E were evaluated in the same manner as in Example 1. Evaluation results are presented in Table 3 and Table 4.

### Comparative Example 2

Immunochromatoassays (testing devices 10) A to E of

Comparative Example 2 were produced in the same manner as in Comparative Example 1, except that the anti-human IgG antibody used in the test-line-reagent coating liquid in Comparative Example 1 was changed to an anti-hCG monoclonal antibody (ANTI-ALPHA SUBUNIT 6601 SPR-5 available from Medix Biochemica Inc.), the human IgG (I2511-10MG available from Sigma-Aldrich Co. LLC) used in the control-line-reagent coating liquid in Comparative Example 1 was changed to an anti-mouse IgG antibody (566-70621 available from Wako Pure Chemical Industries, Ltd.), and the gold-colloid-labeled anti-human IgG antibody used in the labeled-antibody-reagent coating liquid in Comparative Example 1 was changed to the gold-colloid-labeled antibody produced in Example 4.

The produced immunochromatoassays A to E were evaluated in the same manner as in Example 4. Evaluation results are presented in Table 3 and Table 4.

TABLE 3

| | | | Evaluation of visibility | | | | |
|---|---|---|---|---|---|---|---|
| | | Kind of | | B | C | D | E |
| | | covalent | A | Hydrophilic | Hydrophilic | Qualitative | Groundwood |
| | Antibody | binding | Nitrocellulose | PTFE | PVDF | filter | paper |
| Ex. 1 | IgG | Amide | A | A | A | A | A |
| | | binding | | | | | |
| Ex. 2 | IgG | Imine | A | A | A | A | A |
| | | binding | | | | | |
| Ex. 3 | IgG | Thioether | A | A | A | A | A |
| | | bond | | | | | |
| Ex. 4 | hCG | Amide | A | A | A | A | A |
| | | binding | | | | | |
| Comp. | IgG | None | B | C | C | D | D |
| Ex. 1 | | | | | | | |
| Comp. | hCG | None | B | C | C | D | D |
| Ex. 2 | | | | | | | |

TABLE 4

| | Evaluation of density | | | | | | | | | |
|---|---|---|---|---|---|---|---|---|---|---|
| | | | B | | C | | D | | E | |
| | A | | Hydrophilic | | Hydrophilic | | Qualitative | | Groundwood | |
| | Nitrocellulose | | PTFE | | PVDF | | filter | | paper | |
| | Reading | Evaluation | Reading | Evaluation | Reading | Evaluation | Reading | Evaluation | Reading | Evaluation |
| Ex. 1 | 285 | A | 293 | A | 294 | A | 296 | A | 291 | A |
| Ex. 2 | 282 | A | 295 | A | 293 | A | 294 | A | 296 | A |
| Ex. 3 | 294 | A | 280 | A | 284 | A | 282 | A | 286 | A |
| Ex. 4 | 288 | A | 281 | A | 292 | A | 297 | A | 283 | A |
| Comp. | 217 | B | 74 | C | 85 | C | Un-measurable | D | Un-measurable | D |
| Ex. 1 | | | | | | | | | | |
| Comp. | 220 | B | 71 | C | 79 | C | Un-measurable | D | Un-measurable | D |
| Ex. 2 | | | | | | | | | | |

In the evaluation of visibility of Examples 1 to 4, lines having a uniform color optical density throughout the lines and highly visible could be confirmed from any of the immunochromatoassays including the flow path members A to E. In the evaluation of optical density, lines having a high density could be confirmed from any of the immunochromatoassays including the flow path members A to E.

On the other hand, in the evaluation of visibility of Comparative Examples 1 and 2, color development that was not discontinuous as lines could be confirmed from the flow path members A, whereas smudging around the lines were heavy and color development could be barely confirmed from the flow path members B and C. The flow path members D and E resulted in heavy non-specific adsorption to all over the flow path members, and lines could not be confirmed. In the evaluation of optical density, although a density reading higher than 200 was confirmed from the flow path members A, the density was low in the flow path members B and C because the labeling particles in the lines were diffused in the paper and color development was blurred, and color development in the flow path members D and E was so blurred that no line shapes could be confirmed and no density was measurable.

Aspects of the present invention are as follows, for example.

<1> A target testing device including:

a porous flow path member as a flow path for a testing target liquid to flow through;

a first-antibody supplying portion configured to supply a labeled antibody to the testing target liquid flowing through the flow path member, where the labeled antibody contains a label and a first antibody bindable with any target substance that is possibly contained in the testing target liquid; and

a second-antibody immobilized portion that is a resin shaped body disposed at such a position on the flow path as downstream from the first-antibody supplying portion in a manner to contact the flow path member, where a second antibody bindable with at least one of the first antibody and the target substance is immobilized to such a surface of the shaped body as contacting the flow path member by covalent binding with the shaped body.

<2> The target testing device according to <1>,

wherein the covalent binding is at least one selected from amide binding, imine binding, and thioether bond.

<3> The target testing device according <2>, wherein each of the shaped body and the second antibody contains a functional group, the functional group of the shaped body and the functional group of the second antibody being bindable with each other.
<4> The target testing device according to <3>, wherein the functional group of the shaped body is any one of a carboxyl group and an amino group.
<5> The target testing device according to <3> or <4>, wherein the functional group of the second antibody is any one of an amino group at a terminal of the second antibody, a carboxyl group at the terminal of the second antibody, an amino group at a side chain of the second antibody, a carboxyl group at the side chain of the second antibody, and a thiol group at the side chain of the second antibody.
<6> The target testing device according to any one of <1> to <5>, wherein the shaped body is a non-porous body.
<7> A transfer medium for producing a target testing device, the transfer medium intended for producing the target testing device according to any one of <1> to <6>, the transfer medium including:

a support member;

a release layer over the support member; and

a reagent immobilized layer over the release layer,

wherein a reagent reactive with the target substance is immobilized to a surface of the reagent immobilized layer.

<8> A method for producing a testing device, the method including

a step of bringing the reagent immobilized layer of the transfer medium for producing a target testing device according to <7> and the flow path member into contact with each other to transfer the reagent immobilized layer onto the flow path member.

<9> A target testing kit including:

the target testing device according to any one of <1> to <6>; and

an analyte collecting unit configured to collect an analyte.

<10> A target testing method including:

an analyte supplying step of supplying an analyte to the flow path member of the target testing device according to any one of <1> to <6>; and

a step of capturing a part of the analyte by the second antibody immobilized to the shaped body.

What is claimed is:

1. A target testing device comprising:
a porous flow path member as a flow path for a testing target liquid to flow through;
a first-antibody supplying portion configured to supply a labeled antibody to the testing target liquid flowing through the flow path member, where the labeled antibody comprises a label and a first antibody bindable with any target substance possibly comprised in the testing target liquid; and
a second-antibody immobilized portion that is a resin shaped body disposed at a position on the flow path downstream from the first-antibody supplying portion in a manner to contact the flow path member, where a second antibody bindable with at least one of the first antibody and the target substance is immobilized to a surface of the shaped body contacting the flow path member by covalent binding with the shaped body.

2. The target testing device according to claim 1,
wherein the covalent binding comprises at least one selected from amide binding, imine binding, and thioether bond.

3. The target testing device according claim 2,
wherein each of the shaped body and the second antibody comprises a functional group, the functional group of the shaped body and the functional group of the second antibody being bindable with each other.

4. The target testing device according to claim 3,
wherein the functional group of the shaped body comprises any one of a carboxyl group and an amino group.

5. The target testing device according to claim 3,
wherein the functional group of the second antibody comprises any one of an amino group at a terminal of the second antibody, a carboxyl group at the terminal of the second antibody, an amino group at a side chain of the second antibody, a carboxyl group at the side chain of the second antibody, and a thiol group at the side chain of the second antibody.

6. The target testing device according to claim 1,
wherein the shaped body comprises a non-porous body.

7. A transfer medium for producing a target testing device, the transfer medium intended for producing the target testing device according to claim 1, the transfer medium comprising:
a support member;
a release layer over the support member; and
a reagent immobilized layer over the release layer,
wherein a reagent reactive with the target substance is immobilized to a surface of the reagent immobilized layer.

8. A method for producing a target testing device, the method comprising
bringing the reagent immobilized layer of the transfer medium for producing a target testing device according to claim 7 and the flow path member into contact with each other to transfer the reagent immobilized layer onto the flow path member.

9. A target testing kit comprising:
the target testing device according to claim 1; and
an analyte collecting unit configured to collect an analyte.

10. A target testing method comprising:
supplying an analyte to the flow path member of the target testing device according to claim 1; and
capturing a part of the analyte by the second antibody immobilized to the shaped body.
